(19) United States
(12) Patent Application Publication
(10) Pub. No.: US 2026/0243742 A1
(43) Pub. Date: Aug. 20, 2026

(54) THIN-LAYER CHROMATOGRAPHY IDENTIFICATION METHOD FOR CHARACTERISTIC COMPONENTS IN ANSHEN DINGZHI WAN

(71) Applicants: Anhui University of Chinese Medicine, Hefei (CN); Institute of Health and Medicine, Hefei Comprehensive National Science Center, Hefei (CN)

(72) Inventors: Caiyun ZHANG, Hefei (CN); Wenwen TAO, Hefei (CN); Chengjie XING, Hefei (CN); Ting ZHAN, Hefei (CN); Pengfei XIE, Hefei (CN); Yu GAO, Hefei (CN); Xiaoxiao SHAN, Hefei (CN); Can PENG, Hefei (CN); Wei ZHANG, Hefei (CN); Peng HUANG, Hefei (CN)

(73) Assignees: Anhui University of Chinese Medicine, Hefei (CN); Institute of Health and Medicine, Hefei Comprehensive National Science Center, Hefei (CN)

(21) Appl. No.: 19/235,664

(22) Filed: Jun. 12, 2025

(30) Foreign Application Priority Data
Feb. 20, 2025 (CN) 202510188013.4

(51) Int. Cl. G01N 30/94; B01D 15/20; G01N 33/15

(52) U.S. Cl. CPC G01N 30/94; B01D 15/20; G01N 33/15

(57) ABSTRACT

A thin-layer chromatography identification method for characteristic components in Anshen Dingzhi Wan (ASDZW) includes the following steps: S1, spotting: pipetting and spotting a test solution of ASDZW, a negative test solution of ASDZW, a reference medicinal material solution and a reference solution on a silica gel G thin-layer plate; S2, developing: developing the silica gel G thin-layer plate with a developing solvent, taking out the silica gel G thin-layer plate after developing and drying the silica gel G thin-layer plate; and S3, inspection: spraying a chromogenic solution on the dried silica gel G thin-layer plate for performing a chromogenic inspection. The present invention only needs to prepare three test solutions to identify eight characteristic components of ginsenoside Rg1, ginsenoside Rb1, ginsenoside Re, ginsenoside Rf, tenuifolin, dehydrotumulosic acid, pachymic acid and β-asarone in ASDZW, the method has good applicability, clear chromatogram and good separation.

### CROSS REFERENCE TO THE RELATED APPLICATIONS

This application is based upon and claims priority to Chinese Patent Application No. 202510188013.4, filed on Feb. 20, 2025, the entire contents of which are incorporated herein by reference.

### TECHNICAL FIELD

The present invention belongs to the field of pharmaceutical raw material detection technology and traditional Chinese medicine quality control technology, particularly related to a thin-layer chromatography (TLC) identification method for characteristic components in Anshen Dingzhi Wan (ASDZW).

### BACKGROUND

Anshen Dingzhi Wan (ASDZW) is a prescription excerpted from Cheng Guopeng's Medical Mind Comprehension in the Qing Dynasty (Volume 4 • insomnia). ASDZW is composed of poria, poria with hostwood, panax ginseng, polygala tenuifolia, acori tatarinowii rhizoma, and dragon teeth, which are crushed, made into pills with refined honey, and coated with cinnabaris. ASDZW has the effect of tranquilizing and sedating the mind, and strengthening Qi and settling fright, which is a classic prescription used by medical masters in the previous dynasties for the treatment of anxiety, depression, panic and other diseases caused by mental dystrophy.

Thin-layer chromatography is an important experimental technique for rapid separation and qualitative analysis of a small number of substances, the equipment and operation process are relatively simple, with less sample volume and time spent. However, the existing thin-layer chromatography methods are mainly single medicinal materials analysis, there is currently no report on the thin-layer chromatography identification analysis method for the whole prescription of ASDZW. The general practice is to separately identify panax ginseng, polygala tenuifolia, poria, and poria with hostwood in ASDZW, which is difficult to satisfy the requirements of thin-layer chromatography identification efficiency of ASDZW in actual production. Meanwhile, the compound components of ASDZW are complex, and the thin-layer chromatography analysis method of single medicinal material cannot accurately eliminate interference. It is urgent to develop a thin-layer chromatography identification method for the whole prescription of ASDZW.

Components that can be identified by thin-layer chromatography identification in ASDZW mainly include ginsenoside Rb1, ginsenoside Rg1, ginsenoside Re, and ginsenoside Rf from panax ginseng, tenuifolin from polygala tenuifolia, triterpenoids such as pachymic acid and dehydrotumulosic acid from Poria and poria with hostwood, and β-asarone from acori tatarinowii rhizoma. Due to the large structural differences of the above-mentioned characteristic components, existing specific identification methods and conditions are basically adopted for separate identification, which greatly reduces the identification efficiency of the whole prescription of ASDZW. How to achieve efficient identification of multiple characteristic components in ASDZW with relatively simple analysis conditions in the thin-layer chromatography method is of great significance to the identification and quality monitoring of the characteristic components of ASDZW.

### SUMMARY

Based on the technical problems existing in the background technology, the present invention proposes a thin-layer chromatography identification method for characteristic components in Anshen Dingzhi Wan (ASDZW).

A thin-layer chromatography identification method for characteristic components in ASDZW proposed by the present invention, the method includes the following steps:

S1, spotting: pipetting and spotting a test solution of ASDZW, a negative test solution of ASDZW, a reference medicinal material solution, and a reference solution on a silica gel G thin-layer plate;

S2, developing: developing the silica gel G thin-layer plate with a developing solvent, taking out the silica gel G thin-layer plate after developing, and drying the silica gel G thin-layer plate; and

S3, inspection: spraying a chromogenic solution on the dried silica gel G thin-layer plate for performing a chromogenic inspection.

ASDZW includes medicinal materials such as panax ginseng, polygala tenuifolia, poria, poria with hostwood, and acori tatarinowii rhizoma; wherein, the characteristic components in panax ginseng include ginsenoside Rb1, ginsenoside Rg1, ginsenoside Re and ginsenoside Rf; the characteristic components in the polygala tenuifolia includes tenuifolin; and the characteristic components in the poria, poria with hostwood and acori tatarinowii rhizoma include pachymic acid, dehydrotumuloic acid and β-asarone.

Preferably, a thin-layer chromatography identification method for characteristic components of panax ginseng in ASDZW, the method includes the following steps:

S1, spotting: pipetting and spotting a test solution I of ASDZW, a negative test solution I of ASDZW, a reference medicinal material solution I and a reference solution I on a silica gel G thin-layer plate I;

S2, developing: developing the silica gel G thin-layer plate using chloroform-methanol-water as the developing solvent, taking out the silica gel G thin-layer plate after developing, and drying the silica gel G thin-layer plate; and

S3, inspection: spraying a sulfuric acid-ethanol solution on the dried silica gel G thin-layer plate for performing the chromogenic inspection.

More preferably, in the S1, the test solution I of ASDZW is a solution of ASDZW ultrasonically dissolved in 75% ethanol with a mass volume ratio of 1 g/10 mL; the negative test solution I of ASDZW is a solution of panax ginseng-free ASDZW ultrasonically dissolved in 75% ethanol with a mass volume ratio of 2 g/25 mL, the reference medicinal material solution I includes panax ginseng reference medicinal material solution ultrasonically dissolved by 75% ethanol with a mass volume ratio of 1 g/50 mL, respectively; and the reference solution I includes a mixed solution of ginsenoside Rb1 reference, a ginsenoside Rg1 reference, a ginsenoside Re reference and a ginsenoside Rf reference each dissolved in methanol with a mass volume ratio of 1 mg/mL, respectively.

More preferably, in the S1, a spotting volume ratio of the test solution I of ASDZW, the negative test solution I of ASDZW, the reference medicinal material solution I and the reference solution I is 15:15:4:3.

More preferably, in the S2, a volume ratio of chloroform:methanol:water in the chloroform-methanol-water developing solvent is 7:4:(0.5-1.5).

More preferably, in the S3, a mass percentage concentration of the sulfuric acid-ethanol mixed solution is 5%, wherein the volume ratio of the sulfuric acid solution to the ethanol solution is 1:19.

Preferably, a thin-layer chromatography identification method for characteristic components of polygala tenuifolia in ASDZW, the method includes the following steps:

S1, spotting: pipetting and spotting a test solution II of ASDZW, a negative test solution II of ASDZW, a reference medicinal material solution II and a reference solution II on a silica gel G thin-layer plate II;

S2, developing: developing the silica gel G thin-layer plate using chloroform-methanol-water as the developing solvent, taking out the silica gel G thin-layer plate after developing, and drying the silica gel G thin-layer plate; and

S3, inspection: spraying the sulfuric acid-ethanol solution on the dried silica gel G thin-layer plate for performing the chromogenic inspection.

More preferably, in the S1, the test solution II of ASDZW is a solution of ASDZW ultrasonically dissolved in 75% ethanol with a mass volume ratio of 1 g/10 mL; the negative test solution II of ASDZW is a solution of polygala tenuifolia-free ASDZW ultrasonically dissolved in 75% ethanol with a mass volume ratio of 2 g/25 mL, the reference medicinal material solution II is a polygala tenuifolia reference medicinal material solution ultrasonically dissolved in 75% ethanol with a mass volume ratio of 1 g/50 mL; and the reference solution II includes a tenuifolin reference solution dissolved by methanol with a mass volume ratio of 1 mg/mL.

More preferably, in the S1, a spotting volume ratio of the test solution II of ASDZW, the negative test solution II of ASDZW, the reference medicinal material solution II and the reference solution II is 20:20:20:8.

More preferably, in the S2, a volume ratio of chloroform: methanol: water in the chloroform-methanol-water developing solvent is (7-7.5):4:(0.5-1.5).

More preferably, in the S3, a mass percentage concentration of the sulfuric acid-ethanol mixed solution is 5%, wherein the volume ratio of the sulfuric acid solution to the ethanol solution is 1:19.

Preferably, a thin-layer chromatography identification method for characteristic components of poria, poria with hostwood and acori tatarinowii rhizoma in ASDZW, the method includes the following steps:

S1, spotting: pipetting and spotting a test solution III of ASDZW, a negative test solution III of ASDZW, a reference medicinal material solution III and a reference solution III on a silica gel G thin-layer plate III;

S2, developing: developing the silica gel G thin-layer plate using dichloromethane-ethyl acetate as the developing solvent, taking out the silica gel G thin-layer plate after developing, and drying the silica gel G thin-layer plate; and

S3, inspection: spraying a vanillic aldehyde-sulfuric acid-ethanol mixed solution on the dried silica gel G thin-layer plate for performing the chromogenic inspection.

More preferably, in the S1, the test solution III of ASDZW is a solution of ASDZW ultrasonically dissolved by petroleum ether with a mass volume ratio of 1 g/10 mL; the negative test solution III of ASDZW is a solution of ASDZW without poria and poria with hostwood (or acori tatarinowii rhizoma) with a mass volume ratio of (3-4.5) g/50 mL ultrasonically dissolved by petroleum ether; the reference medicinal material solution III includes a solution of the poria or acori tatarinowii rhizoma reference medicinal material with a mass volume ratio of (0.5-1) g/50 mL ultrasonically dissolved by petroleum ether; and the reference solution III included a dehydrotumulosic acid reference solution, a pachymic acid reference solution, and a β-asarone solution each dissolved by methanol with a mass volume ratio of 2 mg/mL.

More preferably, in the S1, a spotting volume ratio of the test solution III of ASDZW, the negative test solution III of ASDZW, the reference medicinal material solution III and the reference solution III is 20:10:10:4.

More preferably, in the S2, a volume ratio of dichloromethane:ethyl acetate in the dichloromethane-ethyl acetate developing solvent is (4-4.5):(1-1.5).

More preferably, in the S3, a mass percentage concentration of the vanillic aldehyde-sulfuric acid-ethanol mixed solution is 2%, wherein a volume ratio of the vanillic aldehyde-sulfuric acid solution to the ethanol solution is 1:9.

Preferably, in the S3, the condition of inspection is inspected in sunlight or under a 365 nm ultraviolet lamp.

The beneficial effects of the present invention are:

the present invention adopts the method of ultrasonic extraction of the solution, and merely requires to prepare three test solutions to effectively identify eight characteristic components of ginsenoside Rb1, ginsenoside Rg1, ginsenoside Re, ginsenoside Rf, tenuifolin, dehydrotumulosic acid, pachymic acid and β-asarone in ASDZW compound prescription; the identification method of the present invention excludes the influence of other components (dragon teeth and cinnabaris) in ASDZW on thin-layer chromatography identification, making the chromatogram clear and the separation good. In the process of thin-layer chromatography analysis, the present invention sets up the negative test solution and the reference medicinal material solution, which enable chromatographic comparison more clearly. Through the test results, it is found that the identification method proposed by this present invention has good applicability, clear chromatograms, and good separation, which significantly improves the success rate of thin-layer chromatography identification of ASDZW compound prescription and meets the requirements of the identification efficiency of ASDZW in actual production. Meanwhile, the present invention inspects and identifies the characteristic components of poria (pachymic acid) and acori tatarinowii rhizoma (β-asarone) in thin-layer chromatography in the 2020 edition of Chinese Pharmacopoeia, and inspects and identifies the thin-layer characteristic components (dehydrotumulosic acid) of poria with hostwood, which are also of great significance for the establishment of quality control indicators of poria, poria with hostwood, and acori tatarinowii rhizoma.

The characteristic components pachymic acid, dehydrotumulosic acid and β-asarone of poria, poria with hostwood, and acori tatarinowii rhizoma in ASDZW compound prescription were detected simultaneously under the condition of one test solution: the ASDZW dispersion solution with a mass volume ratio of (3-4.5) g/50 mL was ultrasonically prepared with petroleum ether as solvent, dichloromethane-ethyl acetate was used as developing solvent to develop the silica gel G thin-layer plate, the silica gel G thin layer plate was taken out and dried after developing, and the vanillic aldehyde-sulfuric acid-ethanol mixed solution was sprayed on the dried silica gel G thin-layer plate for performing the chromogenic inspection. In the chromatographic results of ASDZW, the main spots of pachymic acid, dehydrotumulosic acid, and β-asarone showed the same color at the corresponding positions of the reference medicinal materials, and the separation effect was good.

In the present invention, a single identification method was used to simultaneously identify four ginsenosides in ASDZW, including ginsenoside Rb1, ginsenoside Rg1, ginsenoside Re and ginsenoside Rf, and obtained a clear chromatogram with good separation. In the chromatographic results of the test samples of ASDZW, the main spots of ginsenoside Rb1, ginsenoside Rg1, ginsenoside Re, and ginsenoside Rf showed the same color at the corresponding position of the reference panax ginseng medicinal materials; the separation effect was good, which is simpler and easier to operate than the extraction method and developing solvent of the thin-layer chromatography identification of panax ginseng medicinal materials in the Chinese Pharmacopoeia, and could effectively perform the thin-layer chromatography identification analysis of compound ASDZW.

Meanwhile, the identification method for the characteristic components tenuifolin of polygala tenuifolia in ASDZW compound prescription is also simpler and easier to operate than the extraction method and developing solvent in Chinese Pharmacopoeia, the chromatogram is clear, and the separation is good by controlling the experimental conditions. In the chromatographic results of ASDZW, the main spots of tenuifolin showed the same color at the corresponding position of the reference medicinal materials, the separation effect was good, and the success rate of thin-layer chromatography identification was high, which could be used for the thin-layer chromatography identification of ASDZW. The thin-layer chromatograms inspected in sunlight are still clearly visible, which greatly improves the detection efficiency.

### BRIEF DESCRIPTION OF THE DRAWINGS

FIG. 1A is a thin-layer inspection view of embodiment 1 under sunlight, and FIG. 1B is a thin-layer inspection view of embodiment 1 under an ultraviolet lamp at 365 nm; wherein 1 is a reference solution of ginsenoside Rb1; 2 is a reference solution of ginsenoside Re; 3 is a reference solution of ginsenoside Rf; 4 is a reference solution of ginsenoside Rg1; 5 is a mixed reference solution of ginsenoside Rb1, ginsenoside Re, ginsenoside Rf and ginsenoside Rg1; 6 is a solution of panax ginseng reference medicinal material; 7 is a test solution I of ASDZW; 8 is a negative sample solution I of ASDZW.

FIG. 2 is a thin-layer inspection view of embodiment 2 under sunlight; wherein 1 is a reference solution of tenuifolin; 2 is a solution of polygala tenuifolia reference medicinal materials; 3 is a test solution II of ASDZW; 4 is a negative sample solution II of ASDZW.

FIG. 3 is a thin-layer inspection view of embodiment 3 under sunlight; wherein 1 is a reference solution of dehydrotumulosic acid; 2 is a reference solution of pachymic acid; 3 is a reference solution of β-asarone; 4 is a solution of poria reference medicinal materials; 5 is a solution of acori tatarinowii rhizoma reference medicinal materials; 6 is a test solution III of ASDZW; 7 is a test solution III without poria and poria with hostwood; 8 is a test solution III without acori tatarinowii rhizoma.

FIG. 4A is a thin-layer inspection view of comparative embodiment 1 under sunlight, and FIG. 4B is a thin-layer inspection view of comparative embodiment 1 under an ultraviolet lamp at 365 nm; wherein

1 is a reference solution of ginsenoside Rb1; 2 is a reference solution of ginsenoside Re; 3 is a reference solution of ginsenoside Rf; 4 is a reference solution of ginsenoside Rg1; 5 is a mixed reference solution of ginsenoside Rb1, ginsenoside Re, ginsenoside Rf and ginsenoside Rg1; 6 is a solution of panax ginseng reference medicinal materials; 7 is a test solution I of ASDZW; 8 is a negative sample solution I of ASDZW.

FIG. 5A is a thin-layer inspection view of comparative embodiment 1-2 under sunlight, and FIG. 5B is a thin-layer inspection view of comparative embodiment 1-2 under an ultraviolet lamp at 365 nm; wherein

1 is a reference solution of ginsenoside Rb1; 2 is a reference solution of ginsenoside Re; 3 is a reference solution of ginsenoside Rf; 4 is a reference solution of ginsenoside Rg1; 5 is a mixed reference solution of ginsenoside Rb1, ginsenoside Re, ginsenoside Rf and ginsenoside Rg1; 6 is a solution of panax ginseng reference medicinal materials; 7 is a test solution I of ASDZW; 8 is a negative sample solution I of ASDZW.

FIG. 6A is a thin-layer inspection view of comparative embodiment 1-3 under sunlight, and FIG. 6B is a thin-layer inspection view of comparative embodiment 1-3 under an ultraviolet lamp at 365 nm; wherein

1 is a reference solution of ginsenoside Rb1; 2 is a reference solution of ginsenoside Re; 3 is a reference solution of ginsenoside Rf; 4 is a reference solution of ginsenoside Rg1; 5 is a mixed reference solution of ginsenoside Rb1, ginsenoside Re, ginsenoside Rf and ginsenoside Rg1; 6 is a solution of panax ginseng reference medicinal materials solution; 7 is a test solution I of ASDZW; 8 is a negative sample solution I of ASDZW.

FIG. 7A is a thin-layer inspection view of comparative embodiment 1-4 under sunlight, and FIG. 7B is a thin-layer inspection view of comparative embodiment 1-4 under an ultraviolet lamp at 365 nm; wherein

1 is a reference solution of ginsenoside Rb1; 2 is a reference solution of ginsenoside Re; 3 is a reference solution of ginsenoside Rf; 4 is a reference solution of ginsenoside Rg1; 5 is a mixed reference solution of ginsenoside Rb1, ginsenoside Re, ginsenoside Rf and ginsenoside Rg1; 6 is a panax ginseng reference medicinal materials solution; 7 is a test solution I of ASDZW; 8 is a negative sample solution I of ASDZW.

FIG. 8 is a thin-layer inspection view of comparative embodiment 2-1 under sunlight, wherein

1 is a reference solution of tenuifolin; 2 is a solution of polygala tenuifolia reference medicinal materials; 3 is a test solution II of ASDZW; 4 is a negative sample solution II of ASDZW.

FIG. 9 is a thin-layer inspection view of comparative embodiment 2-2 under sunlight, wherein

1 is a reference solution of tenuifolin; 2 is a solution of polygala tenuifolia reference medicinal materials; 3 is a test solution II of ASDZW; 4 is a negative sample solution II of ASDZW.

FIG. 10 is a thin-layer inspection view of comparative embodiment 2-3 under sunlight, wherein

1 is a reference solution of tenuifolin; 2 is a solution of polygala tenuifolia reference medicinal materials; 3 is a test solution II of ASDZW; 4 is a negative sample solution II of ASDZW.

FIG. 11 is a thin-layer inspection view of comparative embodiment 2-4 under sunlight, wherein

1 is a reference solution of tenuifolin; 2 is a solution of polygala tenuifolia reference medicinal materials; 3 is a test solution II of ASDZW; 4 is a negative sample solution II of ASDZW.

FIG. 12 is a thin-layer inspection view of comparative embodiment 3-1 under sunlight, wherein

1 is a reference solution of dehydrotumulosic acid; 2 is a reference solution of pachymic acid; 3 is a reference solution of β-asarone; 4 is a solution of poria reference medicinal materials; 5 is a reference solution of acori tatarinowii rhizoma reference medicinal materials; 6 is a test solution III of ASDZW; 7 is a test solution III without poria and poria with hostwood; 8 is a test solution III without acori tatarinowii rhizoma.

FIG. 13 is a thin-layer inspection view of comparative embodiment 3-2 under sunlight, wherein

1 is a reference solution of dehydrotumulosic acid; 2 is a reference solution of pachymic acid; 3 is a reference solution of β-asarone; 4 is a solution of poria reference medicinal materials; 5 is a solution of acori tatarinowii rhizoma reference medicinal materials; 6 is a test solution III of ASDZW; 7 is a test solution III without poria and poria with hostwood; 8 is a test solution III without acori tatarinowii rhizoma.

FIG. 14 is a thin-layer inspection view of comparative embodiment 3-3 under sunlight, wherein

1 is a reference solution of dehydrotumulosic acid; 2 is a reference solution of pachymic acid; 3 is a reference solution of β-asarone; 4 is a solution of poria reference medicinal materials; 5 is a solution of acori tatarinowii rhizoma reference medicinal materials; 6 is a test solution III of ASDZW; 7 is a test solution III without poria and poria with hostwood; 8 is a test solution III without acori tatarinowii rhizoma.

FIG. 15 is a thin-layer inspection view of comparative embodiment 3-4 under sunlight, wherein

1 is a reference solution of dehydrotumulosic acid; 2 is a reference solution of pachymic acid; 3 is a reference solution of β-asarone; 4 is a solution of poria reference medicinal materials; 5 is a solution of acori tatarinowii rhizoma reference medicinal materials; 6 is a test solution III of ASDZW; 7 is a test solution III without poria and poria with hostwood; 8 is a test solution III without acori tatarinowii rhizoma.

### DETAILED DESCRIPTION OF THE EMBODIMENTS

The technical solution of the present invention is described in detail by specific embodiments.

The materials, reagents, etc., used in the following embodiments and comparative embodiments are commercially available unless otherwise mentioned.

### Embodiment 1

This embodiment provides a thin-layer chromatography identification method for characteristic components of ginsenoside Rb1, ginsenoside Rg1, ginsenoside Re and ginsenoside Rf of panax ginseng in ASDZW, the method includes the following steps:

Preparation of test solution I of ASDZW: 5 g of ASDZW were taken and finely ground, added 50 mL of 75% ethanol, and ultrasonically treated (power 250 W, and frequency 50 kHz) for 45 min; After filtration, the filtrate was evaporated and dried, and the residue was dissolved with 1 mL of methanol to obtain the test solution I of ASDZW.

Preparation of negative test solution I of ASDZW: 4 g of panax ginseng-free ASDZW was taken, added 50 mL of 75% ethanol, and ultrasonically treated (power 250 W, and frequency 50 kHz) for 45 min; After filtration, the filtrate was evaporated and dried, and the residue was dissolved with 1 mL of methanolto obtain the negative test solution I of ASDZW.

Preparation of reference medicinal material solution I: 1 g of panax ginseng reference medicinal material was taken, and the 1 g/50 mL of panax ginseng reference medicinal material solution was obtained according to the method for preparing the test solution I of ASDZW.

Preparation of reference solution I: 1 mL solution containing 1 mg reference substance was prepared by adding methanol to ginsenoside Rb1 reference substance, ginsenoside Rg1 reference substance, ginsenoside Re reference substance and ginsenoside Rf reference substance, respectively.

Spotting: thin-layer chromatography (Chinese Pharmacopoeia 2020 Edition IV General Principles 0502) test, 15μL of test solution I of ASDZW, 15μL of negative test solution I of ASDZW, 4 μL of reference medicinal material solution I, 3 μL of reference solution I (3 μL each of the reference solution of ginsenoside Rb1, the reference solution of ginsenoside Rg1, the reference solution of ginsenoside Re, and the reference solution of ginsenoside Rf) were pipetted and spotted on silica gel G thin-layer plate I; when spotting, the automatic spotting instrument is used to ensure that the spotting is uniform and neat;

developing: the lower solution of the chloroform-methanol-water mixed solution with a volume ratio of 7:4:1 was used as the developing solvent to place the spotted silica gel G thin-layer plate I in the developing solvent steam, pre-saturated, developed, removed, and dried; and inspection: sulfuric acid-ethanol solution with a mass percentage concentration of 5% (the volume ratio of sulfuric acid solution and ethanol solution is 1:19) was sprayed on the silica gel G thin-layer plate I after drying, heated at 105° C. until the spot color was clear, and inspected under sunlight and ultraviolet light (365 nm).

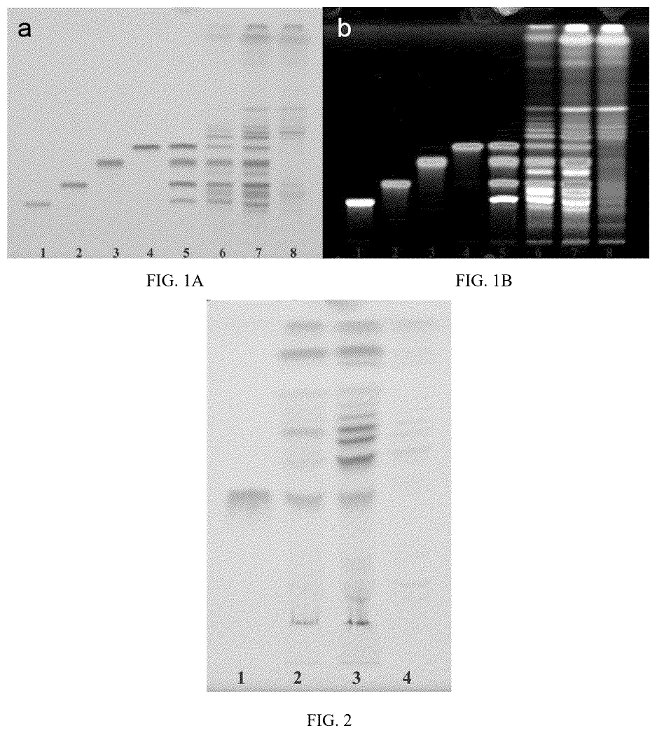

The results are shown in FIGS. 1A-1B, wherein 1 is the reference solution of ginsenoside Rb1; 2 is the reference solution of ginsenoside Re; 3 is the reference solution of ginsenoside Rf; 4 is the reference solution of ginsenoside Rg1; 5 is a mixed reference solution of ginsenoside Rb1, ginsenoside Re, ginsenoside Rf and ginsenoside Rg1; 6 is the solution of panax ginseng reference medicinal material; 7 is the test solution I of ASDZW; 8 is the negative sample solution I of ASDZW. It can be seen that in the chromatogram of ASDZW, the main spots of the same color appeared at the corresponding position of the chromatogram of the reference medicinal materials.

### Embodiment 2

This embodiment provides a thin-layer chromatography identification method for characteristic components of tenuifolin of polygala tenuifolia in ASDZW, the method includes the following steps:

Preparation of test solution II of ASDZW: 5 g of ASDZW was taken, finely ground, placed it in a conical flask with a stopper, added 50 mL of 75% methanol accurately, weighed, and ultrasonically treated (power 400 W, and frequency 40 kHz) for 45 min, shaken well, after filtration, the filtrate was evaporated and dried, 50 mL of water-saturated n-butanol was added to shake and extracte, stood and layered to take the supernatant, evaporated and dried, and the residue was dissolved with 1 mL of methanol to obtain the test solution II of ASDZW.

Preparation of negative test solution II of ASDZW: 4 g of polygala tenuifolia-free ASDZW was taken, finely ground, placed it in the conical flask with the stopper, added 50 mL of 75% methanol accurately, weighed, and ultrasonically treated (power 400 W, and frequency 40 kHz) for 45 min, shaken well, after filtration, the filtrate was evaporated and dried, 50 mL of water-saturated n-butanol was added to shake and extracte, stood and layered to take the supernatant, evaporated and dried, and the residue was dissolved with 1 mL of methanol to obtain the negative test solution II of ASDZW.

Preparation of reference medicinal material solution II: 1 g of polygala tenuifolia reference medicinal material was taken, and the 1 g/50 mL of polygala tenuifolia reference medicinal material solution was obtained according to the method for preparing the test solution II of ASDZW.

Preparation of reference solution II: 1 mL solution containing 1 mg reference substance was prepared by adding methanol to tenuifolin reference substance, that is, the tenuifolin reference solution.

Spotting: thin-layer chromatography (Chinese Pharmacopoeia 2020 Edition IV General Principles 0502) test, 20 μL of test solution II of ASDZW, 20 μL of negative test solution II of ASDZW, 20 μL of reference medicinal material solution II, 8 μL of reference solution II were pipetted and spotted on silica gel G thin-layer plate II; when spotting, the automatic spotting instrument is used to ensure that the spotting is uniform and neat;

developing: the lower solution of the chloroform-methanol-water mixed solution with the volume ratio of 7:4:1 was used as the developing solvent to place the spotted silica gel G thin-layer plate II in the developing solvent steam, pre-saturated, developed, removed, and dried; and

inspection: sulfuric acid-ethanol solution with a mass percentage concentration of 5% (the volume ratio of sulfuric acid solution and ethanol solution is 1:19) was sprayed on the silica gel G thin-layer plate II after drying, heated at 105° C. until the spot color was clear.

The results are shown in FIG. 2, wherein 1 is the reference solution of tenuifolin; 2 is the solution of polygala tenuifolia reference medicinal materials; 3 is the test solution II of ASDZW; 4 is the negative sample solution II of ASDZW. It can be seen that in the chromatogram of the test sample of ASDZW, the main spots of the same color are shown in the corresponding position of the chromatogram of the reference medicinal materials, and the separation effect is good.

### Embodiment 3

This embodiment provides a thin-layer chromatography identification method for characteristic components of dehydrotumuloic acid, pachymic acid, and β-asarone of poria and poria with hostwood in ASDZW, the method includes the following steps:

Preparation of test solution III of ASDZW: 5 g of ASDZW were taken and finely ground, added 50 mL of petroleum ether, and ultrasonically treated (power 250 W, and frequency 50 kHz) for 30 min, after filtration, the filtrate was evaporated and dried, and the residue was dissolved with 1 mL of methanol to obtain the test solution III of ASDZW.

Preparation of negative test solution III of ASDZW: 3 g ASDZW without poria and poria with hostwood was taken, and 4.5 g ASDZW without acori tatarinowii rhizoma were taken and finely ground, added 50 mL of petroleum ether to each, and ultrasonically treate (power 250 W, and frequency 50 kHz) for 30 min, after filtration, the filtrate was evaporated and dried, and the residue was dissolved with 1 mL of methanol to obtain the negative test solution III of ASDZW, that is, the test solution III without poria and poria with hostwood, and the test solution III without acori tatarinowii rhizoma.

Preparation of reference medicinal material solution III: 0.5 g of acori tatarinowii rhizoma reference medicinal material and 1 g of poria reference medicinal material were taken respectively, and the 0.5 g/50 mL of acori tatarinowii rhizoma reference medicinal material solution and 1 g/50 mL of poria reference medicinal material solution were obtained according to the method for preparing the test solution III of ASDZW.

Preparation of reference solution III: 1 mL solution containing 2 mg reference substance was prepared by adding methanol to the dehydrotumulosic acid reference substance, pachymic acid reference substance, β-asarone reference substance, respectively, and the dehydrotumulosic acid reference solution, pachymic acid reference solution and β-asarone reference solution were obtained.

Spotting: thin-layer chromatography (Chinese Pharmacopoeia 2020 Edition IV General Principles 0502) test, 20 μL of test solution III of ASDZW, 10 μL of negative test solution III of ASDZW (10 μL each of the two negative test solution), 10 μL of reference medicinal material solution III (10 μL each of the poria reference medicinal material solution and acori tatarinowii rhizoma reference medicinal material solution), 4 μL of reference solution III (4 μL each of the dehydrotumulosic acid reference solution, pachymic acid reference solution and β-asarone reference solution) were pipetted and spotted on silica gel G thin-layer plate III; when spotting, the automatic spotting instrument is used to ensure that the spotting is uniform and neat;

developing: the dichloromethane-ethyl acetate mixed solution with a volume ratio of 4:1 was used as the developing solvent to place the spotted silica gel G thin-layer plate III in the developing solvent steam, pre-saturated, developed, removed, and dried; and

inspection: vanillic aldehyde-sulfuric acid-ethanol mixed solution with a mass percentage concentration of 2% (the volume ratio of vanillic aldehyde-sulfuric acid solution and ethanol solution is 1:9) was sprayed on the silica gel G thin-layer plate III after drying, heated at 105° C. until the spot color was clear.

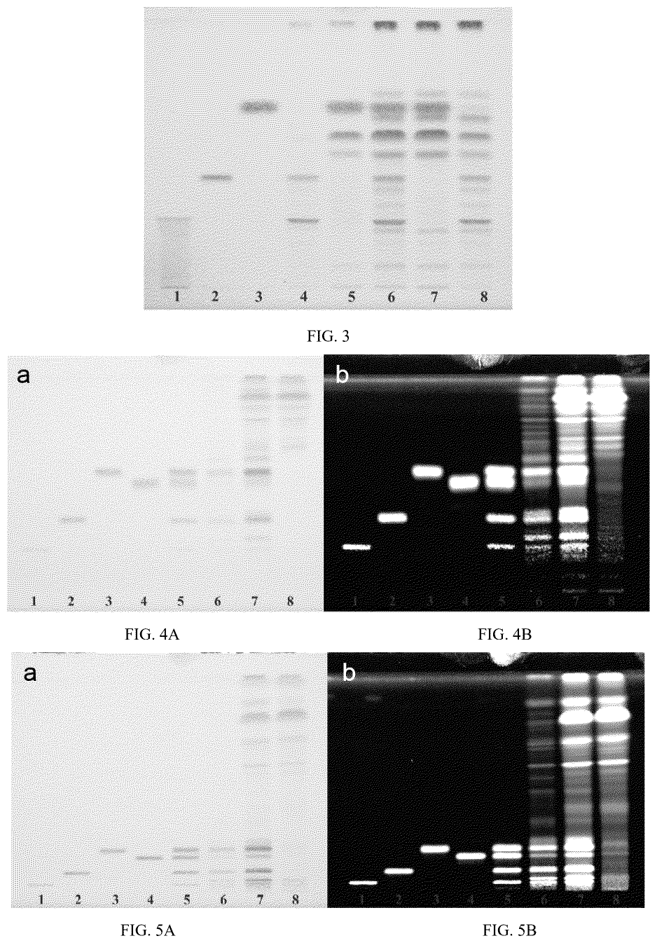

The results are shown in FIG. 3, wherein 1 is the reference solution of dehydrotumulosic acid; 2 is the reference solution of pachymic acid; 3 is the reference solution of β-asarone; 4 is the solution of poria reference medicinal materials; 5 is the solution of acori tatarinowii rhizoma reference medicinal materials; 6 is the test solution II of ASDZW; 7 is the test solution III without poria and poria with hostwood; 8 is the test solution III without acori tatarinowii rhizoma. It can be seen that in the chromatogram of the test sample of ASDZW, the main spots of the same color are shown in the corresponding position of the chromatogram of the reference medicinal materials, and the separation effect is good.

### Comparative Embodiment 1-1

According to the same method of embodiment 1, the thin-layer chromatography identification method for characteristic components of panax ginseng in ASDZW was performed. The difference is that during the developing process, the developing solvent chloroform-methanol-water (7:4:1) was replaced by chloroform-ethyl acetate-methanol-water (15:40:22:10) to develop.

The results are shown in FIGS. 4A-4B, in the chromatogram of ASDZW, the main spots of the same color appeared at the corresponding position of the chromatogram of the reference medicinal materials, but the spots were slightly blurred compared with the developing solvent chloroform-methanol-water (7:4:1), and the separation was not good enough, but it could still be distinguished.

### Comparative embodiment 1-2

According to the same method of embodiment 1, the thin-layer chromatography identification method for characteristic components of panax ginseng in ASDZW was performed. The difference is that during the developing process, the developing solvent chloroform-methanol-water (7:4:1) was replaced by chloroform-methanol-water (7:3:1) to develop.

The results are shown in FIGS. 5A-5B, in the chromatogram of ASDZW, the main spots of the same color appeared at the corresponding position of the chromatogram of the reference medicinal materials, but the spots were slightly blurred compared with the developing solvent chloroform-methanol-water (7:4:1), and the separation was not good enough, but it could still be distinguished.

### Comparative Embodiment 1-3

According to the same method of embodiment 1, the thin-layer chromatography identification method for characteristic components of panax ginseng in ASDZW was performed. The difference is that during the spotting process, 10 μL of test solution I of ASDZW, 10 μL of negative test solution I of ASDZW, 2 μL of reference medicinal material solution I, 2 μL of reference solution I (2 μL each of ginsenoside Rb1 reference solution, ginsenoside Rg1 reference solution, ginsenoside Re reference solution, and ginsenoside Rf reference solution) were pipetted.

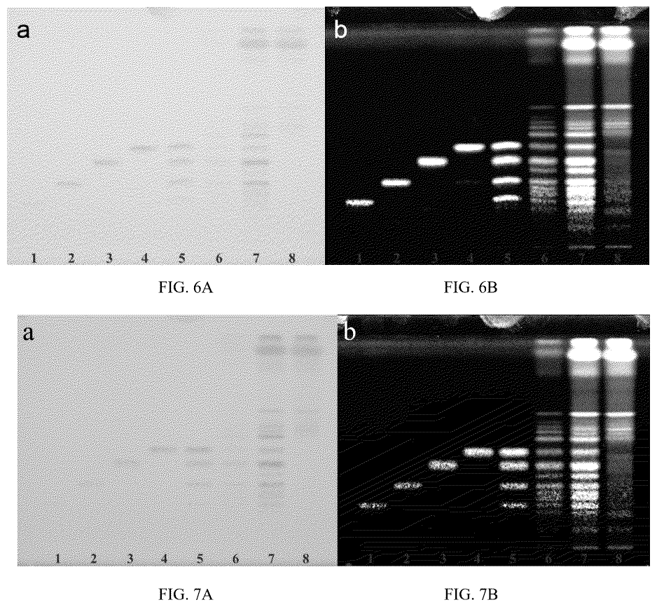

The results are shown in FIGS. 6A-6B, wherein 1 is the reference solution of ginsenoside Rb1; 2 is the reference solution of ginsenoside Re; 3 is the reference solution of ginsenoside Rf; 4 is the reference solution of ginsenoside Rg1; 5 is the mixed reference solution of ginsenoside Rb1,ginsenoside Re, ginsenoside Rf and ginsenoside Rg1; 6 is the solution of panax ginseng reference medicinal materials solution; 7 is the test solution I of ASDZW; 8 is the negative sample solution I of ASDZW. It can be seen that in the chromatogram of ASDZW, the main spots of the same color appeared at the corresponding position of the chromatogram of the reference medicinal materials. It can be seen that in the corresponding position of the chromatogram of the reference medicinal materials, the main spots of the color development are blurred, and the spots are blurred compared with the pipetted 15μL of test solution I of ASDZW, 15 μL of negative test solution I of ASDZW, 4μL of reference medicinal material solution I, 3 μL of reference solution I (3 μL each of ginsenoside Rb1 reference solution, ginsenoside Rg1 reference solution, ginsenoside Re reference solution, and ginsenoside Rf reference solution).

### Comparative Embodiment 1-4

According to the same method of embodiment 1, the thin-layer chromatography identification method for characteristic components of panax ginseng in ASDZW was performed. The difference is that during the developing process, the developing solvent chloroform-methanol-water (7:4:1) was replaced by chloroform-methanol-water (13:7:2) to develop.

The results are shown in FIGS. 7A-7B, when the developing solvent chloroform-methanol-water (7:4:1) was replaced by chloroform-methanol-water (13:7:2) to develop, the color of the spots was blurred.

### Comparative Embodiment 2-1

According to the same method of embodiment 2, the thin-layer chromatography identification method for characteristic components of polygala tenuifolia in ASDZW was performed. The difference is that during the developing process, the developing solvent was the mixed volume ratio of 6:3:0.5 chloroform-methanol-water mixed solution.

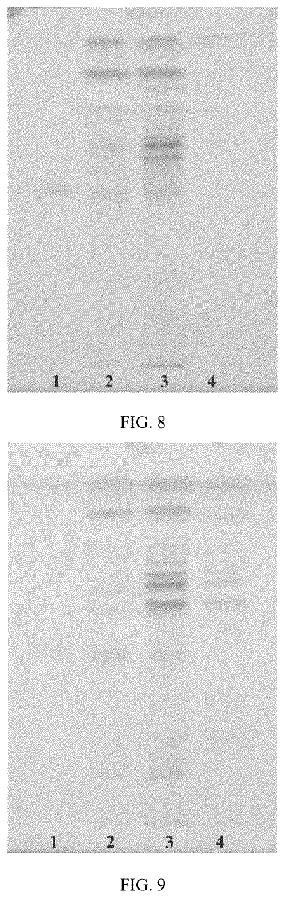

The results are shown in FIG. 8, wherein 1 is the reference solution of tenuifolin; 2 is the solution of polygala tenuifolia reference medicinal materials; 3 is the test solution II of ASDZW; 4 is the negative sample solution II of ASDZW. It can be seen that the developing effect is not ideal, and the main spots of the color development are not clear at the corresponding position of the chromatogram of the reference medicinal materials.

### Comparative Embodiment 2-2

According to the same method of embodiment 2, the thin-layer chromatography identification method for characteristic components of polygala tenuifolia in ASDZW was performed. The difference is that during the developing process, the developing solvent was the mixed volume ratio of 6:4:1 chloroform-methanol-water mixed solution.

The results are shown in FIG. 9, wherein 1 is the reference solution of tenuifolin; 2 is the solution of polygala tenuifolia reference medicinal materials; 3 is the test solution II of ASDZW; 4 is the negative sample solution II of ASDZW. It can be seen that the developing effect is not ideal, and the main spots of the color development are not clear at the corresponding position of the chromatogram of the reference medicinal materials.

### Comparative Embodiment 2-3

According to the same method of embodiment 2, the thin-layer chromatography identification method for characteristic components of polygala tenuifolia in ASDZW was performed. The difference is that during the spotting process, 15 μL of test solution II of ASDZW, 15 μL of negative test solution II of ASDZW, 15 μL of reference medicinal material solution II, and 6 μL of reference solution II were pipetted.

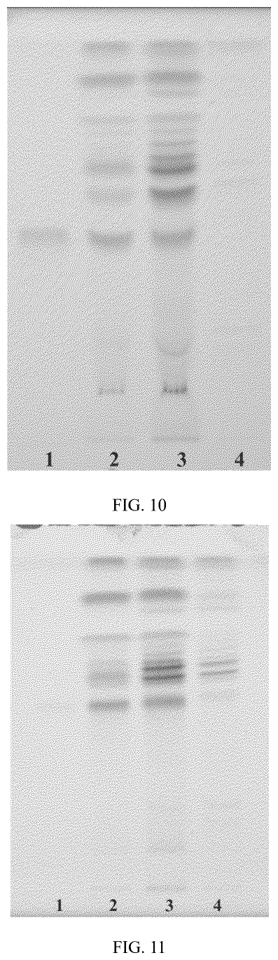

The results are shown in FIG. 10, wherein 1 is the reference solution of tenuifolin; 2 is the solution of polygala tenuifolia reference medicinal materials; 3 is the test solution II of ASDZW; 4 is the negative sample solution II of ASDZW It can be seen that compared with the pipetted 20 μL of test solution II of ASDZW, 20 μL of negative test solution II of ASDZW, 20 μL of reference medicinal material solution II, 8 μL of reference solution II, the main spot of color development is not clear at the corresponding position of the chromatogram of the reference medicinal materials.

### Comparative Embodiment 2-4

According to the same method of embodiment 2, the thin-layer chromatography identification method for characteristic components of polygala tenuifolia in ASDZW was performed. The difference is that during the developing process, the developing solvent was the mixed volume ratio of 15:40:22:10 chloroform-ethyl acetate-methanol-water mixed solution.

The results are shown in FIG. 11, when the developing solvent was the mixed volume ratio of 15:40:22:10 chloroform-ethyl acetate-methanol-water mixed solution, although the color of the spots were clear, the negative test sample was interfered.

### Comparative Embodiment 3-1

According to the same method of embodiment 3, the thin-layer chromatography identification method for characteristic components of poria, poria with hostwood and acori tatarinowii rhizoma in ASDZW was performed. The difference is that during the developing process, the developing solvent was the mixed volume ratio of 20:5:0.5 toluene-ethyl acetate-formic acid mixed solution.

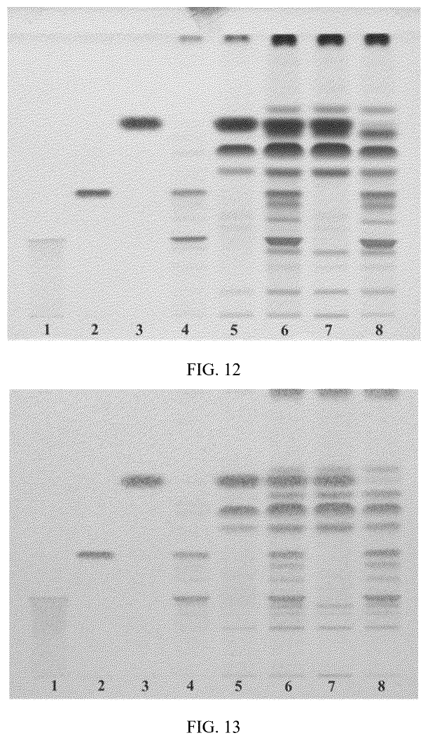

The results are shown in FIG. 12, wherein 1 is the reference solution of dehydrotumulosic acid; 2 is the reference solution of pachymic acid; 3 is the reference solution of β-asarone; 4 is the solution of poria reference medicinal materials; 5 is the reference solution of acori tatarinowii rhizoma reference medicinal materials; 6 is the test solution III of ASDZW; 7 is the test solution III without poria and poria with hostwood; 8 is the test solution III without acori tatarinowii rhizoma. It can be seen that the main spots of the same color appeared at the corresponding position of the chromatogram of the reference medicinal materials, but the color development of the spots was not clear enough, and the separation effect of the β-asarone band in the test sample was not good.

### Comparative Embodiment 3-2

According to the same method of embodiment 3, the thin-layer chromatography identification method for characteristic components of poria, poria with hostwood and acori tatarinowii rhizoma in ASDZW was performed. The difference is that during the developing process, the developing solvent was the mixed volume ratio of 22:5:1 toluene-ethyl acetate-formic acid mixed solution.

The results are shown in FIG. 13, wherein 1 is the reference solution of dehydrotumulosic acid; 2 is the reference solution of pachymic acid; 3 is the reference solution of β-asarone; 4 is the solution of poria reference medicinal materials; 5 is the solution of acori tatarinowii rhizoma reference medicinal materials; 6 is a test solution III of ASDZW; 7 is of β-asaro test solution III without poria and poria with hostwood; 8 is of β-asaro test solution III without acori tatarinowii rhizoma. It can be seen that the main spots of the same color appeared at the corresponding position of the chromatogram of the reference medicinal materials, and the separation effect is better, but the color development of the spots was not clear enough.

### Comparative Embodiment 3-3

According to the same method of embodiment 3, the thin-layer chromatography identification method for characteristic components of poria, poria with hostwood and acori tatarinowii rhizoma in ASDZW was performed. The difference is that during the spotting process, 15 μL of test solution III of ASDZW, 15 μL of negative test solution III of ASDZW (15 μL each of the two negative test solutions), 15 μL of reference medicinal material solution III (15 μL each of the solution of poria reference medicinal material and the solution of acori tatarinowii rhizoma reference medicinal material), 6 μL of reference solution III (5 μL each of the reference solution of dehydrotumulosic acid, the reference solution of pachymic acid and the reference solution of β-asarone) were pipetted.

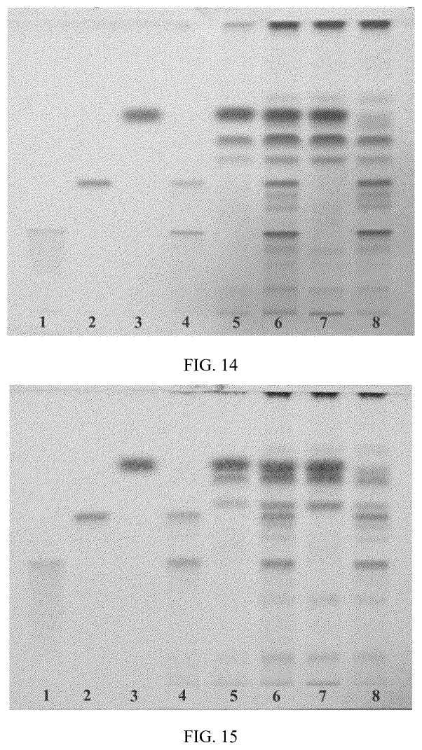

The results are shown in FIG. 14, wherein 1 is the reference solution of dehydrotumulosic acid; 2 is the reference solution of pachymic acid; 3 is the reference solution of β-asarone; 4 is the solution of poria reference medicinal materials; 5 is the solution of acori tatarinowii rhizoma reference medicinal materials; 6 is of β-asaro test solution I of ASDZW; 7 is of β-asaro test solution III without poria and poria with hostwood; 8 is of β-asaro test solution III without acori tatarinowii rhizoma. It can be seen that compared with the pipetted 20 μL of test solution III of ASDZW, 10 μL of negative test solution III of ASDZW (10 μL each of the two negative test solutions), 10 μL of reference medicinal material solution III (10 μL each of the solution of poria reference medicinal material and solution of acori tatarinowii rhizoma reference medicinal material), 4 μL of reference solution III (4 μL each of the reference solution of dehydrotumulosic acid, the reference solution of pachymic acid and the reference solution of β-asarone), the main spots of the same color appeared at the corresponding position of the chromatogram of the reference medicinal materials, but the color development of the spots was not clear enough.

### Comparative Embodiment 3-4

According to the same method of embodiment 3, the thin-layer chromatography identification method for characteristic components of poria, poria with hostwood and acori tatarinowii rhizoma in ASDZW was performed. The difference is that during the developing process, the developing solvent was the mixed volume ratio of 12:3:0.5 dichloromethane-ethyl acetate-formic acid mixed solution.

The results are shown in FIG. 15, when the developing solvent was the mixed volume ratio of 12:3:0.5 dichloromethane-ethyl acetate-formic acid mixed solution, the separation of β-asarone in the test sample is not good, and the color is blurred.

In summary, the thin-layer chromatography identification method for characteristic components in ASDZW proposed by the present invention has good applicability, the obtained chromatogram is clear, and the separation is good, which significantly improves the success rate of thin-layer chromatography identification of ASDZW and meets the requirements of the identification efficiency of ASDZW in actual production.

The above is merely a preferred specific embodiment of the present invention, but the scope of protection of the present invention is not limited thereto, and any skilled person familiar with the technical field of the present invention shall be equivalently replaced or changed according to the technical scheme of the present invention and its invention conception within the technical scope disclosed by the present invention, which should be covered within the scope of protection of the present invention.

What is claimed is:

1. A thin-layer chromatography (TLC) identification method for characteristic components in Anshen Dingzhi Wan (ASDZW), comprising the following steps:
S1, a spotting: pipetting and spotting a test solution of the ASDZW, a negative test solution of the ASDZW, a reference medicinal material solution, and a reference solution on a silica gel G thin-layer plate;
S2, a developing: developing the silica gel G thin-layer plate with a developing solvent, taking out a developed silica gel G thin-layer plate after the developing, and drying the developed silica gel G thin-layer plate to obtain a dried silica gel G thin-layer plate; and
S3, an inspection: spraying a chromogenic solution on the dried silica gel G thin-layer plate for performing a chromogenic inspection;
wherein traditional Chinese medicinal materials for a thin-layer chromatography identification in the ASDZW comprise panax ginseng, polygala tenuifolia, poria, poria with hostwood, and acori tatarinowii rhizoma; wherein characteristic components in the panax ginseng comprise ginsenoside Rb1, ginsenoside Rg1, ginsenoside Re, and ginsenoside Rf; characteristic components in the polygala tenuifolia comprise tenuifolin; and characteristic components in the poria, the poria with hostwood, and the acori tatarinowii rhizoma comprise pachymic acid, dehydrotumuloic acid, and β-asarone;
in the step S3, the inspection is conducted in a sunlight or under a 365 nm ultraviolet lamp.

2. The thin-layer chromatography identification method according to claim 1, wherein in the thin-layer chromatography identification method for the characteristic components of the panax ginseng in the ASDZW, the developing solvent is chloroform-methanol-water, and the chromogenic solution is a sulfuric acid-ethanol solution.

3. The thin-layer chromatography identification method according to claim 2, wherein in the step S1, the test solution of the ASDZW is a solution of the ASDZW ultrasonically dissolved in 75% ethanol with a mass volume ratio of 1 g/10 mL; the negative test solution of the ASDZW is a solution of panax ginseng-free ASDZW ultrasonically dissolved in the 75% ethanol with a mass volume ratio of 2 g/25 mL, and the reference medicinal material solution comprises a panax ginseng reference medicinal material solution ultrasonically dissolved in the 75% ethanol with a mass volume ratio of 1 g/50 mL; and the reference solution comprises a mixed solution of a ginsenoside Rb1 reference, a ginsenoside Rg1 reference, a ginsenoside Re reference, and a ginsenoside Rf reference each dissolved in methanol with a mass volume ratio of 1 mg/mL, respectively.

4. The thin-layer chromatography identification method according to claim 2, wherein in the step S1, a spotting volume ratio of the test solution of the ASDZW, the negative test solution of the ASDZW, the reference medicinal material solution, and the reference solution is 15:15:4:3; in the step S2, a volume ratio of chloroform: methanol: water in the developing solvent is 7:4:(0.5-1.5); and in the step S3, a mass percentage concentration of the sulfuric acid-ethanol solution is 5%, wherein a volume ratio of a sulfuric acid solution to an ethanol solution is 1:19.

5. The thin-layer chromatography identification method according to claim 1, wherein in the thin-layer chromatography identification method for the characteristic components of the polygala tenuifolia in the ASDZW, the developing solvent is chloroform-methanol-water, and the chromogenic solution is a sulfuric acid-ethanol solution.

6. The thin-layer chromatography identification method according to claim 5, wherein in the step S1, the test solution of the ASDZW is a solution of the ASDZW ultrasonically dissolved in 75% ethanol with a mass volume ratio of 1 g/10 mL; the negative test solution of the ASDZW is a solution of polygala tenuifolia-free ASDZW ultrasonically dissolved in the 75% ethanol with a mass volume ratio of 2 g/25 mL, and the reference medicinal material solution is a polygala tenuifolia reference medicinal material solution ultrasonically dissolved in the 75% ethanol with a mass volume ratio of 1 g/50 mL; and the reference solution comprises a tenuifolin reference solution dissolved by methanol with a mass volume ratio of 1 mg/mL.

7. The thin-layer chromatography identification method according to claim 5, wherein in the step S1, a spotting volume ratio of the test solution of the ASDZW, the negative test solution of the ASDZW, the reference medicinal material solution, and the reference solution is 20:20:20:8; in the step S2, a volume ratio of chloroform: methanol: water in the developing solvent is (7-7.5): 4: (0.5-1.5); and in the step S3, a mass percentage concentration of the sulfuric acid-ethanol solution is 5%, wherein a volume ratio of a sulfuric acid solution to an ethanol solution is 1:19.

8. The thin-layer chromatography identification method according to claim 1, wherein in the thin-layer chromatography identification method for the characteristic components of the poria, the poria with hostwood, and the acori tatarinowii rhizoma in the ASDZW, the developing solvent is dichloromethane-ethyl acetate, and the chromogenic solution is a vanillic aldehyde-sulfuric acid-ethanol mixed solution.

9. The thin-layer chromatography identification method according to claim 8, wherein in the step S1, the test solution of the ASDZW is a solution of the ASDZW ultrasonically dissolved by petroleum ether with a mass volume ratio of 1 g/10 mL; the negative test solution of the ASDZW is a solution of the ASDZW without the poria, the poria with hostwood, and the acori tatarinowii rhizoma with a mass volume ratio of (3-4.5) g/50 mL ultrasonically dissolved by the petroleum ether; the reference medicinal material solution comprises a solution of a poria reference medicinal material or an acori tatarinowii rhizoma reference medicinal material with a mass volume ratio of (0.5-1) g/50 mL ultrasonically dissolved by the petroleum ether; and the reference solution comprises a dehydrotumulosic acid reference solution, a pachymic acid reference solution, and a β-asarone solution each dissolved by methanol with a mass volume ratio of 2 mg/mL.

10. The thin-layer chromatography identification method according to claim 8, wherein in the step S1, a spotting volume ratio of the test solution of the ASDZW, the negative test solution of the ASDZW, the reference medicinal material solution, and the reference solution is 20:10:10:4; in the step S2, a volume ratio of dichloromethane: ethyl acetate in the developing solvent is (4-4.5): (1-1.5); and in the step S3, a mass percentage concentration of the vanillic aldehyde-sulfuric acid-ethanol mixed solution is 2%, wherein a volume ratio of a vanillic aldehyde-sulfuric acid solution to an ethanol solution is 1:9.
